(19) United States
(12) Patent Application Publication
(10) Pub. No.: US 2010/0136237 A1
(43) Pub. Date: Jun. 3, 2010

(54) SOLVENT FOR CLEANING OF ORGANIC THIN FILM

(75) Inventors: Kazuhisa Kumazawa, Ichihara (JP); Mikiya Shimada, Chiba (JP)

Correspondence Address: OLIFF & BERRIDGE, PLC, P.O. BOX 320850, ALEXANDRIA, VA, 22320-4850, US

(73) Assignee: NIPPON SODA CO., LTD., Tokyo (JP)

(21) Appl. No.: 12/452,170

(22) PCT Filed: Jul. 4, 2008
(86) PCT No.: PCT/JP2008/001786

§ 371 Date: Dec. 18, 2009

(30) Foreign Application Priority Data
Jul. 5, 2007 (JP) 2007-177341

(51) Int. Cl. C07C 15/02; B05D 3/10

(52) U.S. Cl. 427/331; 585/24

(57) ABSTRACT

It is to provide a cleaning solvent capable of forming a thin film with little falling off of the film component. It is a solvent for cleaning an organic thin film having a solubility of 100 to 400 mg/g at 25° C. for a polymer obtained by subjecting n-octadecyltrimethoxysilane to a hydrolytic polycondensation with KOH. The solvent is preferably an aromatic hydrocarbon solvent comprising at least one compound represented by the formula (I) (wherein each R may be the same or different and represents C₁-C₁₈ alkyl group, and n represents 2, 3 or 4), where diethylbenzene or Solvesso (registered trademark) is particularly preferred.

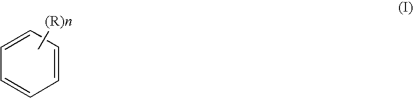

### TECHNICAL FIELD

The present invention relates to an organic solvent that is suitable for washing an organic thin film.

### BACKGROUND ART

Conventionally in various fields, the surface of a substrate consisting of glass, metal, plastic, ceramics and the like are modified in accordance with the intended purpose. For example, a silane coupling agent is coated onto the surface of glass or plastics to confer water-repellency and oil-repellency.

A step of coating a silane coupling agent is exemplified by the following process.

After washing an untreated substrate with pure water, alcohols, etc., the substrate surface is activated by a method such as UV/ozone treatment. Subsequently, the substrate is immersed in a tank containing a silane coupling agent to coat the silane coupling agent onto the substrate. After taking the substrate out from the immersing tank, the substrate is washed with an organic solvent and dried.

Here, the washing step is necessary in order to remove excess organic solvent solution or impurities remained on the substrate surface and to obtain an organic thin film with better film properties such as heat resistance and durability. Also, film thickness can be controlled by washing.

Examples of the solvent used for washing include paraffins, aromatic hydrocarbons, alicyclic hydrocarbons, halogen compounds, and ketones. Specifically used are pentane, hexane, heptane, octane, isooctane, benzene, toluene, xylene, cyclohexane, ligroin, petroleum ether, chloroform, methylene chloride, acetone, and the like (Patent Documents 1 and 2, Non-patent Document 1, etc.).

A washing method using a solvent is not particularly limited as long as the method enables to remove extraneous matter from the substrate surface that has been contacted with a thin film forming solution. Specifically exemplified is a method in which the substrate which has been contacted with an organic thin film forming solution is immersed in a solvent, or a method in which a hydrocarbon organic solvent is sprayed.

However, it has not been possible to obtain a thin film with no falling off of the film component by using such as hexane or xylene.

Patent Document 1: Japanese Laid-Open Patent Application No. 2006-283011

Patent Document 2: Japanese Laid-Open Patent Application No. 2004-91503

Non-patent Document 1: Langmuir 2000, 16, 3932-3936

### DISCLOSURE OF THE INVENTION

### Object to be Solved by the Invention

The present invention was made in view of the circumstances as above. The purpose of the present invention is to form an organic thin film that causes little falling off of the film component as compared to when the conventional methods mentioned above are employed.

### Means to Solve the Object

The present inventors have dedicated to solve the object as above and discovered that an organic thin film that causes little falling off of the film component can be formed by selecting a particular organic solvent. The present invention was thus completed.

The present invention is related to the following:

(1) A cleaning solvent for an organic thin film having a solubility of 100 to 400 mg/g at 25° C. for a polymeric substance obtained by subjecting n-octadecyltrimethoxysilane to a hydrolytic polycondensation with KOH;
(2) The cleaning solvent for an organic thin film according to (1), wherein the organic thin film is formed from an organosilane compound;
(3) The cleaning solvent for an organic thin film according to (1) or (2), wherein the solvent is an aromatic hydrocarbon solvent comprising at least one compound represented by the formula (I)

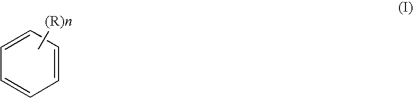

(wherein each R may be the same or different and represents C₁-C₁₈ alkyl group, and n represents 2, 3 or 4);
(4) The cleaning solvent for an organic thin film according to any one of (1) to (3), wherein the solvent is diethylbenzene or Solvesso (registered trademark);
(5) A method for producing an organic thin film comprising the steps of:
(a) contacting a substrate with a solution for forming an organic thin film containing a metal surfactant which has at least one or more hydrolyzable group or hydroxyl group and a catalyst which can interact with the metal surfactant, to form an organic thin film on the surface of the substrate; and
(b) subsequently washing the substrate with the cleaning solvent for an organic thin film according to any one of (1) to (4).

### BEST MODE OF CARRYING OUT THE INVENTION

### (1) Cleaning Solvent

The cleaning solvent for an organic thin film of the present invention is used for removing excess component of the organic thin film or excess organic thin film layer after the organic thin film has been formed on a substrate.

The cleaning solvent for an organic thin film of the present invention is not particularly limited as long as it can adequately dissolve organosilane compounds and the cross-linked substances thereof shown below that form an organic thin film.

In the present invention, a cleaning solvent that can adequately dissolve organosilane compounds and the cross-linked substances thereof needs to meet the following requirements:

1) To have a solvency power such that an excess layer of the organic thin films laminated on the substrate can be removed.

2) Not to have a solvency power that is too strong, in order that it can be avoided that the organic thin film component is dissolved too much to the extent to dissolve a part of the film bound to the substrate and to cause falling off of the film.

As a solvent that meets the above requirements, a solvent is preferred which confers solubility of 100 to 400 mg/g at 25° C. for a polymeric substance (an aggregate of polymeric substances having the maximum molecular weight of about 4,000 when measured with MALDI-TOFMS) obtained by dissolving 35 g of n-octadecyltrimethoxysilane in 1 L methanol, adding thereto 20 g of 0.2N KOH, subjecting the resultant to a hydrolytic polycondensation at room temperature for about 2 weeks, then filtering, washing and drying the generated precipitate.

As an organic solvent that satisfies the above conditions, there is an aromatic hydrocarbon compound represented by the following formula (I).

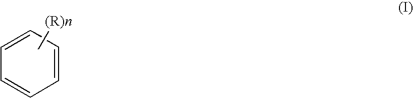

(In the formula, each R may be the same or different and represents C₁-C₁₈ alkyl group, and n represents 2, 3 or 4. Note that when n is 2, a case where both Rs are methyl group shall be excluded.)

Examples of the compounds encompassed by the above formula (I) include 1,2-diethylbenzene, 1,3-diethylbenzene, 1,4-diethylbenzene, 1,2-dimethyl-4-ethylbenzene, 1,3-dimethyl-5-ethylbenzene, 1,4-dimethyl-2-ethylbenzene, 1,2,3,5-tetramethylbenzene, 1,2-dipropylbenzene, 1,2-dibutylbenzene, 1,2-dihexylbenzene, 1,2-didecylbenzene, and 1,2-dioctadecylbenzene. One of these compounds, or 2 or more of these compounds are used.

Preferred compounds among these are benzene compounds substituted by C₁-C₄ alkyl groups, for example, diethylbenzene, Solvesso, and so on.

Diethylbenzene may be any one of o-, m- and p-, and a mixture of these may also suffice.

Solvesso (registered trademark) is a solvent manufactured by Exxon Mobil Corporation, and is an aromatic hydrocarbon solvent containing dialkylbenzene and trialkylbenzene. Solvesso is commercially available as Solvesso 100, Solvesso 150 and Solvesso 200.

Solvesso 150 used in the Examples of the present invention contains following components. 1,2-dimethyl-4-ethylbenzene, 1,3-dimethyl-5-ethylbenzene, 1,4-dimethyl-2-ethylbenzene, 1,3-dimethyl-4-ethylbenzene, 1,2-dimethyl-3-ethylbenzene, 1,3-diethylbenzene, 1,2,3,5-tetramethylbenzene, 1,2,4,5-tetramethylbenzene, 1,2,3-trimethylbenzene, etc.

In the present invention, measurement of the above-mentioned solubility is conducted as follows.

To a solvent (1 g), a polymeric substance of n-octadecyltrimethoxysilane is added while stirring with a spatula and dissolved at room temperature (25° C.). When dissolution no longer proceeds at room temperature, dissolution was allowed to proceed by treating in an ultrasonic washer for 1 min, and the solubility is calculated based on the dissolved amount.

### (2) Organic Thin Film

The organic thin film referred to in the present invention is a thin film consisting of a metal surfactant having a hydrophobic group such as a hydrocarbon group, and includes both monomolecular film and multilayer film. The present invention is particularly suitable for production of monomolecular films. It may also be a self-aggregating film. A self-aggregating film herein means a film generated by forming an ordered structure without any external compelling force.

The production method for an organic thin film using the cleaning solvent of the present invention is explained in the following.

An organic thin film of the present invention is produced by the steps of contacting, in an organic solvent, a substrate with a solution for forming an organic thin film containing a metal surfactant which has at least one or more hydrolyzable group or hydroxyl group and a catalyst which can interact with the metal surfactant.

Details of the mechanism in which a metal surfactant is adsorbed onto the surface of a substrate are not clear, but it can be considered as follows when a substrate having an active hydrogen on the surface is used. Namely, in a solution for forming an organic thin film, a hydrolyzable group in the metal surfactant is in a hydrolyzed state by water. The metal surfactant in this state reacts with an active hydrogen on the substrate surface and a thin film generated by forming a strong chemical bond with the substrate is thus formed.

There is no particular limitation to an amount of a metal surfactant to be contained in the solution for forming an organic thin film used in the present invention. It is, however, preferred that the metal surfactant is contained in an amount range of 0.1 to 30% by mass in order to produce a dense monomolecular film.

Further, although the usage of a catalyst that can interact with a metal surfactant is not particularly limited as long as physical properties of the monomolecular organic thin film formed are not affected, the catalyst is usually used in a range of 0.001 to 1 moles, preferably 0.001 to 0.2 moles, in terms of converted number of oxide moles relative to 1 mole of the metal surfactant.

Organic solvents, metal surfactants, catalysts, substrates and the like used herein are explained below.

### (Metal Surfactant)

There is no particular limitation to a metal surfactant contained in a solution for forming an organic thin film and having at least one or more hydrophobic group or hydroxyl group, as long as at least one or more hydrolysable functional group or hydroxyl group, and a hydrophobic group are comprised in the same molecule, but those metal surfactants are preferred that comprise a hydrolyzable group or a hydroxyl group that can form a bond through a reaction with the active hydrogen on the substrate surface. Such metal surfactant is preferably exemplified by a compound represented by the formula (II).

R¹ₙMXₘ₋ₙ (II)

In the formula: R¹ represents a hydrocarbon group optionally having a substituent or a hydrocarbon group having a linking group; M represents at least one metal atom selected from the group consisting of a silicon atom, germanium atom, tin atom, titanium atom and zirconium atom; X represents a hydroxyl group or a hydrolyzable group; n represents any integer from 1 to (m−1); and m represents the atomic valence of M. When n is 2 or more, R¹ may be the same or different, and when (m−n) is 2 or more, X may be the same or different. Note that among (m−n)Xs, at least one X is a hydrolyzable group or a hydroxyl group.

Examples of the hydrocarbon group optionally having a substituent include: an alkyl group having 1 to 30 carbons such as a methyl group, ethyl group, n-propyl group, isopropyl group, n-butyl group, isobutyl group, sec-butyl group, t-butyl group, n-pentyl group, isopentyl group, neopentyl group, t-pentyl group, n-hexyl group, isohexyl group, n-heptyl group, n-octyl group and n-decyl group; an alkenyl group having 2 to 30 carbons such as a vinyl group, propenyl group, butenyl group and pentenyl group; and an aryl group such as a phenyl group and naphthyl group.

Examples of the substituent of the hydrocarbon group optionally having a substituent include: a carboxyl group; an amido group; an imido group; an ester group; an alkoxy group such as a methoxy group and ethoxy group; a halogen atom such as a fluorine atom and chlorine atom; and a hydroxyl group. Preferred number of these substituents is 0 to 3.

Specific examples of the hydrocarbon group having a linking group are the same as those groups exemplified for the hydrocarbon group optionally having a substituent.

It is preferred that the linking group is present between the carbon-carbon bond of the hydrocarbon group, or between a carbon of the hydrocarbon group and the metal atom M which is described in the following.

Specific examples of the linking group include: —O—, —S—, —SO₂—, —CO—, —C(═O)O—, or —C(═O)NR⁵¹— (wherein R⁵¹ represents a hydrogen atom; or an alkyl group such as methyl group, ethyl group, n-propyl group and isopropyl group).

Among these, R¹ is preferred to be an alkyl group having 1 to 30 carbons, more preferably an alkyl group having 10 to 25 carbons, from the view point of water-repellency and durability.

M represents one kind of atom selected from the group consisting of a silicon atom, germanium atom, tin atom, titanium atom and zirconium atom. Particularly preferred among these is a silicon atom from the view point of easy obtainability, reactivity, etc.

X represents a hydroxyl group or a hydrolyzable group. A hydrolyzable group is not particularly limited as long as it is a group that is degraded through a reaction with water. Specific examples include: a hydrocarbonoxy group optionally having a substituent; an acyloxy group optionally having a substituent; a halogen atom such as a fluorine atom, chlorine atom, bromine atom and iodine atom; an isocyanate group; a cyano group; an amino group; and an amide group.

Particularly preferred among the above are the following groups: a hydroxycarbonoxy group such as an alkoxy group optionally having a substituent, cycloalkoxy group optionally having a substituent, alkenyloxy group optionally having a substituent, aryloxy group optionally having a substituent and aralkyloxy group optionally having a substituent; and an acyloxy group such as an acetoxy group optionally having a substituent.

Examples of the alkoxy group include methoxy group, ethoxy group, n-propoxy group, isopropoxy group, n-butoxy group, sec-butoxy group, t-butoxy group, n-pentyloxy group and n-hexyloxy group, among which an alkoxy group having 1 to 6 carbons is preferred.

Examples of the alkenylxoy group include a vinyloxy group, allyloxy group and 3-n-butenyloxy group, where an alkenyloxy group having 2 to 6 carbons is preferred.

Examples of the cycloalkoxy group include a cyclopropyloxy group, cyclopentyloxy group and cyclohexyloxy group, where a cycloalkoxy group having 3 to 8 carbons is preferred.

Examples of the aryloxy group include a phenyloxy group, naphthyloxy group, azulenyloxy group, indenyloxy group; indanyloxy group and tetralinyloxy group, where an aryloxy group having 6 to 10 carbons is preferred.

Examples of the aralkyloxy group include a benzyloxy group, phenethyloxy group and 1-phenyl-n-hexoxy group, where an aralkyloxy group having 6 to 10 carbons is preferred.

Examples of the acyloxy group include: an alkylcarbonyloxy group such as an acetoxy group, propionyloxy group, n-propylcarbonyloxy group, isopropylcarbonyloxy group and n-butylcarbonyloxy group; a cycloalkylcarbonyloxy group such as a cyclopropylcarbonyloxy group, cyclopropylmethylcarbonyloxy group and cyclohexylcarbonyloxy group; an alkenylcarbonyloxy group such as an acryloyloxy group and allylcarbonyloxy group; and an arylcarbonyloxy group such as a benzoyloxy group.

Examples of the above substituents include a carboxyl group, amide group, imide group, ester group and hydroxyl group. Among these, it is preferred that X is a hydroxyl group, halogen atom, alkoxy group having 1 to 6 carbons, acyloxy group or isocyanate group, where an alkoxy group having 1 to 4 carbons or acyloxy group is more preferred.

m represents the atomic valence of a metal atom M.

n represents any integer from 1 to (m−1). It is preferred that n is 1 for producing a high density organic thin film.

When n is 2 or more, each R¹ may be the same or different.

Further, when (m−n) is 2 or more, X may be the same or different, but at least one X among (m−n)Xs is a hydrolyzable group or a hydroxyl group.

Specific examples of the metal surfactant shown by the formula (II) include the following. In the following, the compounds whose metal atom is a silicon atom is shown as representative examples, but the present invention shall not be limited to these examples.

Namely, followings are exemplified, without being limited to these: CH₃(CH₂)₅Si(OCH₃)₃, CH₃(CH₂)₇Si(OCH₃)₃, CH₃(CH₂)₉Si(OCH₃)₃, CH₃(CH₂)₁₁Si(OCH₃)₃, CH₃(CH₂)₁₃Si(OCH₃)₃, CH₃(CH₂)₁₅Si(OCH₃)₃, CH₃(CH₂)₁₇Si(OCH₃)₃, CH₃(CH₂)₁₉Si(OCH₃)₃, CH₃(CH₂)₂₁Si(OCH₃)₃, CH₃(CH₂)₁₇Si(OCH₂CH₃)₃, CH₃(CH₂)₁₇SiCl₃, CH₃(CH₂)₉Si(OCH₂CH₃)₃, CH₃(CH₂)₉SiCl₃, CH₃(CH₂)₉Si(CH₃)(OCH₂CH₃)₂, CH₃(CH₂)₉Si(CH₃)(OCH₃)₂, CH₃(CH₂)₉Si(CH₃)₂(OCH₂CH₃), CH₃(CH₂)₉Si(CH₃)₂(OCH₃), CH₃CH₂O(CH₂)₁₅Si(OCH₃)₃, CH₃(CH₂)₂Si(CH₃)₂(CH₂)₁₅Si(OCH₃)₃, CH₃(CH₂)₆Si(CH₃)₂ (CH₂)₉Si(OCH₃)₃, CH₃COO(CH₂)₁₅Si(OCH₃)₃, CH₃CH₂O(CH₂)₁₅Si(OC₂H₅)₃, CH₃(CH₂)₂Si(CH₃)₂(CH₂)₁₅Si (OC₂H₅)₃, CH₃(CH₂)₆Si(CH₃)₂(CH₂)₉Si(OC₂H₅)₃, CH₃COO(CH₂)₁₅Si (OC₂H₅)₃, CH₃CH₂O(CH₂)₁₅Si (OCH₃)(OH)₂, CH₃(CH₂)₂Si(CH₃)₂(CH₂)₁₅Si(OCH₃)(OH)₂, CH₃(CH₂)₆Si(CH₃)₂(CH₂)₉Si(OCH₃)(OH)₂, CH₃COO(CH₂)₁₅Si(OCH₃)(OH)₂, CH₃CH₂O(CH₂)₁₅Si(OC₂H₅)(OH)₂, CH₃(CH₂)₂Si(CH₃)₂(CH₂)₁₅Si(OC₂H₅)(OH)₂, CH₃(CH₂)₆Si(CH₃)₂(CH₂)₉Si(OC₂H₅)(OH)₂, CH₃COO(CH₂)₁₅Si(OC₂H₅)(OH)₂, CH₃CH₂O(CH₂)₁₅Si(OCH₃)₂(OH), CH₃(CH₂)₂Si(CH₃)₂(CH₂)₁₅Si(OCH₃)₂(OH), CH₃(CH₂)₆Si(CH₃)₂(CH₂)₉Si(OCH₃)₂(OH), CH₃COO(CH₂)₁₅Si (OCH₃)₂(OH), CH₃CH₂O(CH₂)₁₅Si (OC₂H₅)₂ (OH), CH₃(CH₂)₂Si(CH₃)₂(CH₂)₁₅Si(OC₂H₅)₂(OH), CH₃(CH₂)₆Si(CH₃)₂(CH₂)₉Si(OC₂H₅)₂(OH), CH₃COO(CH₂)₁₅Si(OC₂H₅)₂(OH), CH₃(CH₂)₇Si(OCH₃)(OH)₂, CH₃(CH₂)₇Si(OCH₃)₂(OH), CH₃CH₂O(CH₂)₁₅Si(OH)₃, CH₃(CH₂)₂Si(CH₃)₂(CH₂)₁₅Si(OH)₃, CH₃(CH₂)₆Si(CH₃)₂(CH₂)₉Si(OH)₃, CH₃COO(CH₂)₁₅Si(OH)₃, CH₃CH₂O(CH₂)₁₅Si(OH)₃, CH₃(CH₂)₂Si(CH₃)₂(CH₂)₁₅Si(OH)₃, CH₃(CH₂)₆Si(CH₃)₂(CH₂)₉Si(OH)₃, CH₃COO(CH₂)₁₅Si(OH)₃, CH₃(CH₂)₇Si(OH)₃.

These compounds may be used alone or by combining two or more kinds thereof.

(Catalyst that can Interact with a Metal Surfactant)

The catalyst which is contained in a solution for forming an organic thin film of the present invention and which can interact with the metal surfactant is not particularly limited as long as it is a catalyst that activates a hydrolyzable group or a hydroxyl group by the interaction with the metal moiety or the hydrolyzable group moiety, etc. of a metal surfactant through such as a coordinate bond or a hydrogen bond, and that has a condensation promoting function. Preferred among these are at least one compound selected from the group consisting of: metal oxide; metal hydroxide; metal alkoxides; a chelated or coordinated metal compound; a partially hydrolyzed product of metal alkoxides; a hydrolytic product obtained by treating metal alkoxides with water of two-fold or more equivalent amounts; organic acid; a silanol condensation catalyst; and acid catalyst, and more preferred are metal akloxides and a partially hydrolyzed product of metal alkoxides.

### (Organic Solvent Used for a Solution for Forming an Organic Thin Film)

An organic solvent to be used for a solution for forming an organic thin film of the present invention is preferably an organic solvent wherein the hydrolyzed products of metal alkoxides can be dispersed as a dispersed material in that organic solvent. A solvent having high solubility to water and not causing clotting at a low temperature is more preferred, since a reaction of treating a metal surfactant with water can be conducted at a low temperature.

Specific examples of the organic solvent used include: an alcoholic solvent such as methanol, ethanol and isopropanol; a halogenated hydrocarbonic solvent such as methylene chloride, chloroform and chlorobenzene; a hydrocarbonic solvent such as hexane, cyclohexane, benzene, toluene and xylene; an ether solvent such as tetrahydrofuran, diethylether and dioxane; a ketone solvent such as acetone, methylethylketone and methylisobutylketone; an amido solvent such as dimethylformamide and N-methylpyrrolidone; a sulfoxide solvent such as dimethylsulfoxide; and silicone such as methylpolysiloxane, octamethylcyclotetrasiloxane, decamethylcyclopentanesiloxane and methylphenylpolysiloxane (Japanese Laid-Open Patent Application No. 9-208438).

These solvents can be used alone or by combining two or more kinds thereof.

When using as a mixed solvent, combination of a hydrocarbonic solvent such as toluene and xylene, and a lower alcoholic solvent such as methanol, ethanol, isopropanol and t-butanol is preferred. Herein, as a lower alcoholic solvent, it is more preferred to use secondary or higher alcoholic solvents, such as isopropanol and t-butanol. Mixing ratio of the mixed solvent is not particularly limited, but it is preferred to use a hydrocarbonic solvent and a lower alcoholic solvent in a range of 99/1 to 50/50 as a volume ratio.

### (Substrate)

A substrate used for a method for producing an organic thin film of the present invention is not particularly limited with regard to its material, form and the like. However, a substrate having on its surface a functional group which can interact with molecules that form an organic thin film in the organic solvent solution of the present invention is preferred, and a substrate having an active hydrogen on its surface is particularly preferred. By using a substrate having an active hydrogen on the surface, a chemical adsorption film can be formed easily on the substrate surface by a chemical interaction between the active hydrogen on the substrate surface and the molecules in the organic solvent solution of the present invention.

An active hydrogen is referred to an hydrogen that tend to dissociate as a proton, and a functional group containing an active hydrogen is exemplified by a hydroxyl group (—OH), carboxyl group (—COOH), formyl group (—CHO), imino group (═NH), amino group (—NH₂) and thiol group (—SH), among which a hydroxyl group is preferred.

Specific examples of the substrate having a hydroxyl group on the substrate surface include substrates made from: a metal such as aluminum, copper and stainless; glass; silicon wafer; ceramics; paper; natural fiber; leather; other hydrophilic materials; etc.

A substrate consisting of a material not having a hydroxyl group on the surface, such as plastics and synthetic fibers, can also be preferably used by treating the substrate surface in advance in a plasma atmosphere containing oxygen (e.g., 20 min at 100 w) or by introducing a hydrophilic group by a corona treatment. A substrate made from polyamide resin, polyurethane resin or the like has an imino group on its surface, and an active hydrogen of the imino group and an alkoxysilyl group, etc. of a metal surfactant cause a de-alcohol reaction to form a siloxane bond (—SiO—) . Therefore, surface treatment is not particularly necessary.

Preferred substrate for use in the method for producing an organic thin film of the present invention is a substrate composed of at least one material selected from metal, glass, silicon wafer, ceramics and plastics.

Further, when a substrate not having an active hydrogen on the surface is used, it is also possible to form a silica underlayer having an active hydrogen on the surface by contacting at least one compound selected from SiCl₄, SiHCl₃, SiH₂Cl₂, Cl—(SiCl₂O)_{b}—SiCl₃ (wherein b is a positive integer) with the surface of the substrate, then by allowing a dehydrochlorination reaction to occur.

(Method for Contacting a Solution for Forming an Organic Thin Film with a Substrate)

A method for contacting a solution for forming an organic thin film with a substrate is not particularly limited and known methods can be used. Specific examples include, dipping method, spin coating method, spraying method, roller coat method, Mayer bar method, screen printing method, and brush coating method. Among these, dipping method is preferred. A step of contacting a solution for forming an organic thin film with a substrate may be carried out once for a long period of time or may be carried out by conducting coating in a short time for plural times. Ultrasonic may also be used to promote film formation.

### (3) Method for Washing an Organic Thin Film

After an organic thin film is formed on a substrate, excess component of the organic thin film or excess organic thin film layer on the substrate are washed and removed.

Washing methods include the following:

1) Washing the substrate by pouring a cleaning solution thereto, conducting an ultrasonic treatment in the cleaning solution, washing again by pouring the cleaning solution if necessary, and finally heating and drying the substrate;
2) Washing the substrate by pouring a cleaning solution thereto, conducting a heat treatment, conducting an ultrasonic treatment in the cleaning solution, washing again by pouring the cleaning solution if necessary, and finally heating and drying the substrate;
3) Washing the substrate by pouring a cleaning solution thereto, then heating and drying the substrate.

Here, washing by pouring means pouring a cleaning solution onto the substrate by using a shower or the like.

The heat treatment in the method of “2)” above is usually conducted at 60 to 120° C. for 5 to 30 min.

An ultrasonic treatment in the cleaning solution is usually conducted for 15 to 60 sec. The substrate may be pulled out after the ultrasonic device is halted. But, by pulling out the substrate while the ultrasonic device is in operation, reattachment of the organic thin film component dissolved in the solvent can be minimized.

Heating and drying are usually conducted at 50 to 80° C. for 5 to 20 min.

The present invention will now be described in detail by way of examples, but the technical scope of the present invention is not limited to these exemplifications.

### EXAMPLE

### (1) Preparation of a Solution for Forming an Organic Thin Film

16.1 g (43.0 mmol) of octadecyltrimethoxysilane (Gelest: 95% purity) was put in a 200 mL four-neck flask at room temperature, 4.6 g (16.4 mmol) of tetraisopropoxy titanium (Nippon Soda Co. Ltd.) was added thereto, and diluted by addition of 77.6 g of toluene.

To this solution, 1.7 g of distilled water was added and reacted for 24 hours at room temperature to obtain the solution A.

Next, 78.9 g (200 mmol) of octadecyltrimethoxysilane (Gelest: 95% purity) was put in a 1000 mL four-neck flask at room temperature, 0.16 g of the solution A was added thereto, and diluted by addition of 419 g of toluene.

To this solution, 3.7 g of distilled water was added and reacted for 10 days at room temperature to obtain the solution B.

Thereafter, 20 g of the solution B was put in a 1000 mL four-neck flask at room temperature, and diluted by addition of 480 g of toluene to obtain a solution for forming an organic thin film.

### (2) Production of an Organic Thin Film

Pre-washing was carried out by ultrasonic washing using pure water and alcohol, and further, the 2-inch wafer that had been subjected to UV/ozone treatment for 10 min was immersed in the solution for forming an organic thin film for 10 min. The wafer was pulled out and subjected to the next washing.

### (3) Washing

Washing test was conducted by using various cleaning solvents shown in the table below.

TABLE 1

| | | | ODS polymer |
|---|---|---|---|
| | Solvent | Composition | solubility |
| Example | Diethylbenzene | Aromatic | 300 |
| Example | Solvesso 150 | Aromatic | 350 |
| Comparative | AQUA Solvent G | Paraffin | <20 |
| Example | | | |
| Comparative | Xylene | Aromatic | 550 |
| Example | | | |

### Note)

AQUA Solvent (registered trademark) G is a solvent manufactured by AQUA CHEMICAL CO., LTD.

ODS polymer solubility is a solubility of a polymer at 25° C., wherein the polymer is obtained by dissolving 35 g of n-octadecyltrimethoxysilane (ODS) in 1 L methanol, adding thereto 20 g of 0.2NKOH, subjecting the resultant to a hydrolytic polycondensation at room temperature for about 2 weeks, then filtering, washing and drying the generated precipitate.

### [Washing Example 1]

Washing was carried out as follows by using Solvesso 150, diethylbenzene, xylene and AQUA Solvent G.

2-inch wafer on which an organic thin film had been formed was washed by pouring 100 ml of cleaning solvent followed by heating at 80° C. for 10 min. Subsequently, the 2-inch wafer was immersed in a container containing 1 L of cleaning solvent and ultrasonically treated for 30 sec. The 2-inch wafer was pulled out from the cleaning solvent and heated and dried at 80° C. for 10 min.

### [Washing Example 2]

Washing was carried out as follows by using Solvesso 150 and xylene.

2-inch wafer on which an organic thin film had been formed was washed by pouring 100 ml of cleaning solvent followed by heating at 60° C. for 10 min. Subsequently, the 2-inch wafer was immersed in a container containing 1 L of cleaning solvent and ultrasonically treated for 30 sec. The 2-inch wafer was pulled out from the cleaning solvent and heated and dried at 60° C. for 20 min.

### [Washing Example 3]

Washing was carried out as follows by using Solvesso 150, diethylbenzene and xylene.

2-inch wafer on which an organic thin film had been formed was washed by pouring 100 ml of cleaning solvent followed by heating at 60° C. for 10 min. Subsequently, the 2-inch wafer was immersed in a container containing 1 L of cleaning solvent and ultrasonically treated for 30 sec.

The 2-inch wafer was pulled out from the cleaning solvent and heated and dried at 60° C. for 20 min.

### (4) Evaluation

### 1) Micro Evaluation

Plural sites on the surface of wafers that had been washed with the above solvents were observed under AFM (Atomic Force Microscope).

It was confirmed from the photographs taken that the wafers washed with Solvesso 150 and diethylbenzene showed no falling off of the film and that a uniform organic thin film was achieved. On the other hand, the wafer washed with xylene was confirmed to result in falling off of the film depending on the conditions. Further, when a wafer was washed with AQUA Solvent, particles remained on the wafer due to an insufficient detergency.

### 2) Evaluation by Eye Observation

The surface of wafers that had been washed with the above solvents were irradiated with light and observed.

Only little uneven washing was seen for wafers washed with Solvesso 150 and diethylbenzene. On the other hand, uneven washing was observed for the wafer washed with xylene due to the falling off of the film. Further, when a wafer was washed with AQUA Solvent, many bright spots remained due to an insufficient detergency.

### INDUSTRIAL APPLICABILITY

A cleaning solvent for an organic thin film of the present invention enables to form an organic thin film that causes little falling off of the film component.

1. A cleaning solvent for an organic thin film having a solubility of 100 to 400 mg/g at 25° C. for a polymeric substance obtained by subjecting n-octadecyltrimethoxysilane to a hydrolytic polycondensation with KOH.

2. The cleaning solvent for an organic thin film according to claim 1, wherein the organic thin film is formed from an organosilane compound.

3. The cleaning solvent for an organic thin film according to claim 1, wherein the solvent is an aromatic hydrocarbon solvent comprising at least one compound represented by the formula (I) 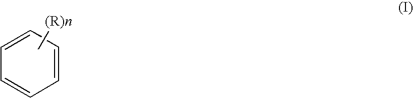
wherein each R may be the same or different and represents C₁-C₁₈ alkyl group, and n represents 2, 3 or 4.

4. The cleaning solvent for an organic thin film according to claim 1, wherein the solvent is diethylbenzene or an aromatic hydrocarbon solvent containing dialkylbenzene and trialkylbenzene.

5. A method for producing an organic thin film comprising the steps of:
(a) contacting a substrate with a solution for forming an organic thin film containing a metal surfactant which has at least one or more hydrolyzable group or hydroxyl group and a catalyst which can interact with the metal surfactant, to form an organic thin film on the surface of the substrate; and
(b) subsequently washing the substrate with the cleaning solvent for an organic thin film according to claim 1.

6. The cleaning solvent for an organic thin film according to claim 2, wherein the solvent is an aromatic hydrocarbon solvent comprising at least one compound represented by the formula (I) 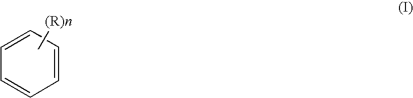
wherein each R may be the same or different and represents C₁-C₁₈ alkyl group, and n represents 2, 3 or 4.

7. The cleaning solvent for an organic thin film according to claim 2, wherein the solvent is diethylbenzene or an aromatic hydrocarbon solvent containing dialkylbenzene and trialkylbenzene.

8. A method for producing an organic thin film comprising the steps of:
(a) contacting a substrate with a solution for forming an organic thin film containing a metal surfactant which has at least one or more hydrolyzable group or hydroxyl group and a catalyst which can interact with the metal surfactant, to form an organic thin film on the surface of the substrate; and
(b) subsequently washing the substrate with the cleaning solvent for an organic thin film according to claim 2.

9. A method for producing an organic thin film comprising the steps of:
(a) contacting a substrate with a solution for forming an organic thin film containing a metal surfactant which has at least one or more hydrolyzable group or hydroxyl group and a catalyst which can interact with the metal surfactant, to form an organic thin film on the surface of the substrate; and
(b) subsequently washing the substrate with the cleaning solvent for an organic thin film according to claim 3.

10. A method for producing an organic thin film comprising the steps of:
(a) contacting a substrate with a solution for forming an organic thin film containing a metal surfactant which has at least one or more hydrolyzable group or hydroxyl group and a catalyst which can interact with the metal surfactant, to form an organic thin film on the surface of the substrate; and
(b) subsequently washing the substrate with the cleaning solvent for an organic thin film according to claim 4.
